(19) United States
(12) Patent Application Publication
(10) Pub. No.: US 2023/0355494 A1
(43) Pub. Date: Nov. 9, 2023

(54) SHAVING COMPOSITION

(71) Applicant: FALLIEN COSMECEUTICALS, LTD., Norristown, PA (US)

(72) Inventors: David J. MILORA, Blue Bell, PA (US); Harry FALLICK, Wayne, PA (US)

(21) Appl. No.: 18/245,739

(22) PCT Filed: Sep. 27, 2021
(86) PCT No.: PCT/US2021/052176

§ 371 Date: Mar. 17, 2023

Related U.S. Application Data
(60) Provisional application No. 63/084,640, filed on Sep. 29, 2020.

(51) Int. Cl. A61K 8/44; A61Q 9/02; A61K 8/46; A61K 8/41

(52) U.S. Cl. CPC A61K 8/442; A61Q 9/02; A61K 8/466; A61K 8/416; A61K 2800/596; A61K 2800/87

(57) ABSTRACT

The invention relates to shaving compositions that are non-fatty acid-soap based, but still provide the desired lubrication for shaving. In embodiments, the shaving compositions include mixtures of amphoteric and anionic surfactants, along with other components such as, e.g., humectant, moisturizing agent, chelating agent, preservative, natural extract, opacifier, foaming agent, fragrance, and water.

### FIELD OF THE INVENTION

The invention relates to shaving compositions that are non-fatty acid-soap based, but still provide the desired lubrication for shaving. In embodiments, the shaving compositions include mixtures of amphoteric and anionic surfactants, along with other components such as, e.g., humectant, moisturizing agent, chelating agent, preservative, natural extract, opacifier, foaming agent, fragrance, and water.

### BACKGROUND

Shaving foams are generally in the form of an oil-in-water emulsion, wherein the water phase contains a soap, e.g., a fatty acid-based soap. Such shaving foams can cause dryness and irritation and/or increase the roughness of the skin, especially sensitive or broken skin, due to the presence of soap in the formula. Soap-containing shaving foams can also cause premature dulling of razor blades. However, soap-free formulations include drawbacks such as difficulty in producing foams with consistent texture and with satisfactory rigidity.

### SUMMARY OF THE INVENTION

The present disclosure relates to shaving compositions.

In some embodiments, the disclosure provides a shaving composition comprising:
a) about 1% w/w to about 50% w/w of an amphoteric surfactant;
b) about 0.1% w/w to about 20% w/w of an anionic surfactant;
c) about 0.1% w/w to about 20% w/w of a humectant;
d) 0% to about 15% w/w of a moisturizing agent;
e) optionally one or more of a moisturizing agent, a chelating agent, a preservative, a natural extract, an opacifier, a fluorinated foaming agent, and a fragrance; and
f) the remainder water,
wherein the composition has a pH of about 3.5 to about 8, and
wherein the composition comprises less than 1% w/w of a fatty acid.

In some embodiments, the shaving composition comprises about 2.5% w/w to about 40% w/w of the amphoteric surfactant. In further embodiments, the amphoteric surfactant comprises an amphoacetate, an amphodiacetate, a betaine, a sarcosinate, a hydroxysultaine, or a combination thereof. In still further embodiments, the shaving composition comprises about 2% w/w to about 10% w/w of an amphodiacetate. In still further embodiments, the shaving composition comprises about 0.5% w/w to about 10% w/w of a betaine. In still further embodiments, the shaving composition comprises about 0.1% w/w to about 10% w/w of a sarcosinate. In certain embodiments, the shaving composition comprises about 2% w/w to about 10% w/w of an amphodiacetate, about 0.5% w/w to about 10% w/w of an amidopropyl betaine, and about 0.1% w/w to about 10% w/w of lauroyl sarcosinate.

In some embodiments, the shaving composition comprises about 0.5% w/w to about 15% w/w of an anionic surfactant. In further embodiments, the anionic surfactant comprises a sulfonate, a sulfate, an ether sulfate, an ether sulfonate, an ether carboxylate, an isethionate, a taurate, an amino acid surfactant, or a combination thereof. In still further embodiments, the shaving composition comprises about 1% w/w to about 10% of an olefin sulfonate. In yet further embodiments, the olefin sulfonate is C₁₂₋₁₄ olefin sulfonate, C₁₄₋₁₆ olefin sulfonate, C₁₄₋₁₈ olefin sulfonate, C₁₆₋₁₈ olefin sulfonate, or a combination thereof.

In some embodiments, the shaving composition further comprises a cationic component. In additional embodiments, the composition has a pH of about 5.5 to about 7.0.

In some embodiments, the shaving composition comprises less than about 1% w/w of each of a cellulose or a natural polymer; a silicone oil, polymer, or copolymer thereof; a mineral, vegetable, or sebaceous oil; and a gelling agent. In further embodiments, the shaving composition comprises less than 0.1% w/w of each of the fatty acid; the cellulose or the natural polymer; the silicone oil, polymer, or copolymer thereof, the mineral, vegetable, or sebaceous oil; and the gelling agent.

In some embodiments, the disclosure provides a shaving composition comprising:
a) about 1% w/w to about 15% w/w of an amphodiacetate;
b) about 0.1% w/w to about 15% w/w of an amidopropyl betaine;
c) about 0.05% w/w to about 15% w/w of lauroyl sarcosinate;
d) about 0.5% w/w to about 15% w/w of an olefin sulfonate;
e) about 0.05% w/w to about 20% w/w of a humectant;
f) 0% to about 5% w/w of a moisturizing agent;
g) optionally one or more of a chelating agent; a preservative; a natural extract, an opacifier, a fluorinated foaming agent, and a fragrance; and
h) the remainder water,
wherein the composition has a pH of about 5.5 to about 7.5; and
wherein the composition comprises less than 1% w/w of each of: a fatty acid; a cellulose or a natural polymer; a silicone oil, polymer, or copolymer thereof, a mineral, vegetable, or sebaceous oil; and a gelling agent.

In some embodiments, the shaving composition comprises about 2% w/w to about 10% w/w of the amphodiacetate. In further embodiments, the shaving composition comprises about 0.5% w/w to about 10% w/w of the amidopropyl betaine. In still further embodiments, the shaving composition comprises about 0.1% w/w to about 10% w/w of lauroyl sarcosinate. In yet further embodiments, the shaving composition comprises about 1% w/w to about 10% w/w of the olefin sulfonate.

In some embodiments, the shaving composition comprises less than about 0.1% of each of the fatty acid; the cellulose or the natural polymer; the silicone oil, polymer, or copolymer thereof; the mineral, vegetable, or sebaceous oil; and the gelling agent.

In some embodiments, the disclosure provides a shaving composition consisting of:
a) about 1% w/w to about 50% w/w of an amphoteric surfactant;
b) about 0.1% w/w to about 20% w/w of an anionic surfactant;
c) about 0.1% w/w to about 20% w/w of a humectant;
d) one or more of a moisturizing agent, a chelating agent, a preservative, and a natural extract, an opacifier, a fluorinated foaming agent, and a fragrance; and
e) the remainder water,
wherein the composition has a pH of about 5.5 to about 7.5.

In some embodiments, the amphoteric surfactant is about 2.5% w/w to about 40% w/w of the composition. In further embodiments, the anionic surfactant is about 1% w/w to about 10% w/w of the composition. In still further embodiments, the humectant is about 0.1% to about 15% w/w of the composition.

In some embodiments, the disclosure provides a method of dispensing the shaving composition described herein, comprising:
a. providing the shaving composition in a gas-in-can dispenser, a finger-actuated dispenser, an aerosol dispenser, or a bag-on-valve dispenser; and
b. dispensing the shaving composition.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each were specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y, and any numbers that fall within x and y.

Unless otherwise indicated, all weight percentages (% w/w) herein refer to the weight of the specified component relative to total weight of the composition, where the total amount of all components in the composition equals 100% by weight (100 weight %). Unless otherwise indicated, the specified amount for a component refers to the combined percentage of all substances that fall within the definition of such component. For example, “about 50% of an amphoteric surfactant” means that the combined percentage of all substances in the composition that are “amphoteric surfactants” as defined herein, is about 50%.

As described herein, the soap-free shaving compositions can produce foams of satisfactory rigidity and consistency and leave the skin lubricious enough during application and after shaving such that shaving razors and devices do not require self-lubricating mechanisms attached to the device during the shaving process. Such soap-free shaving products provide the benefits of substantially less drying and irritation of the skin as compared to soap-containing shaving products. Soap-free shaving products can also slow the dulling of razor blades compared to soap-containing shaving products, which leads to lower costs to the consumer and reduced waste.

As used herein, “soap-free” is understood as meaning containing less than 5% w/w of a fatty acid, more suitably less than 3% of a fatty acid, less than 2% of a fatty acid, and in suitable embodiments, less than 1% w/w of a fatty acid in the composition. In further embodiments, the shaving composition described herein comprises less than 0.5% w/w of a fatty acid, preferably less than 0.1% w/w of a fatty acid. Fatty acids are compounds that consist of a carboxylic acid (—COOH) and an aliphatic chain of at least six carbons, for example, medium-chain fatty acids (MCFA) containing about 6 to about 12 carbons, or long-chain fatty acids (LCFA) containing about 13 to about 21 carbons, or very long chain fatty acids (VLCFA) containing more than 22 carbons. The fatty acid may be saturated (i.e., does not contain any carbon-carbon double bonds) or unsaturated (i.e., containing at least one carbon-carbon double bond). Exemplary fatty acids that are excluded from the present shaving compositions include, but are not limited to, myristic acid, palmitic acid, stearic acid, caprylic acid, capric acid, lauric acid, arachidic acid, behenic acid, lignoceric acid, and cerotic acid.

Fatty acids described herein are distinguished from fatty acid derivatives, which can contain a modification of the carboxylic acid on a fatty acid and/or an additional functional group besides a carboxylic acid. Examples of fatty acid derivatives include, but are not limited to, sarcosinates, triglycerides, phospholipids, sphingolipids, glycolipids, fatty amides, fatty esters, fatty acid chlorides, ether carboxylates, and the like. Unless otherwise indicated, the shaving compositions described herein (i.e., that do not comprise a fatty acid) may contain a fatty acid derivative.

In some embodiments, the disclosure provides a shaving composition comprising:
a) about 1% w/w to about 50% w/w of an amphoteric surfactant;
b) about 0.1% w/w to about 20% w/w of an anionic surfactant;
c) about 0.1% w/w to about 20% w/w of a humectant;
d) about 0% to about 15% w/w of a moisturizing agent;
e) optionally one or more of a moisturizing agent, a chelating agent, a preservative, a natural extract, an opacifier, a fluorinated foaming agent, and a fragrance; and
f) the remainder water,
wherein the composition has a pH of about 3.5 to about 8, and
wherein the composition comprises less than 1% w/w of a fatty acid.

A “surfactant” herein refers to a compound that lowers the surface tension between two liquids. In general, surfactants are amphiphilic compounds that include both a hydrophobic “tail” group and a hydrophilic “head” group, which is positively or negatively charged.

Suitably, the shaving composition described herein comprises an amphoteric surfactant. As used herein, an “amphoteric surfactant,” also known as “zwitterionic surfactant,” means a surfactant that includes both a positive charge (cationic group) and a negative charge (anionic group) on its hydrophilic head group. In some embodiments, the cationic group comprises an amine. In some embodiments, the anionic group comprises a sulfonate, a sulfate, a phosphate, or a combination thereof.

In embodiments, the shaving composition comprises about 0.5% w/w to about 60% w/w, about 1% w/w to about 50% w/w, about 2% w/w to about 45% w/w, about 2.5% w/w to about 40% w/w, about 3% w/w to about 35% w/w, or about 5% w/w to about 30% w/w of the amphoteric surfactant. Suitably, the shaving composition comprises about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w, about 35% w/w, about 40% w/w, about 45% w/w, about 50% w/w, about 55% w/w, or about 60% w/w of the amphoteric surfactant, including all values and ranges within these ranges.

Non-limiting examples of amphoteric surfactants include amphoacetates such as, e.g., sodium lauryl amphoacetate and other alkylamphoacetates; amphodiacetates such as, e.g., disodium cocoamphodiacetate; betaines such as, e.g., alkylbetaines, N-alkylamidobetaines, coco betaine, and cocamidopropyl betaine; sarcosinates such as, e.g., lauroyl sarcosinate, cocoyl sarconsinate, myristoyl sarcosinates, oleoyl sarcosinates; hydroxysultaines such as, e.g., cocamidopropyl hydroxysultaine, alkylpolyaminocarboxylates (APAC); 3-[(3-cholamidopropyl)dimethylammonio]-1-propanesulfonate (CHAPS); and the like. In some embodiments, the amphoteric surfactant of the shaving composition described herein comprises an amphoacetate, an amphodiacetate, a betaine, a sarcosinate, a hydroxysultaine, or a combination thereof.

Suitably, the shaving composition comprises about 1% w/w to about 20% w/w, preferably about 1.5% w/w to about 15% w/w, or more preferably about 2% w/w to about 10% w/w of an amphodiacetate. In some embodiments, the shaving composition comprises about 1% w/w, about 1.5% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 15% w/w, or about 20% w/w of an amphodiacetate, including all values and ranges within these ranges. In some embodiments, the amphodiacetate is disodium cocoamphodiacetate.

Suitably the shaving composition comprises about 0.1% w/w to about 20% w/w, preferably about 0.25% to about 15% w/w, or more preferably about 0.5% w/w to about 10% w/w of a betaine. In some embodiments, the shaving composition comprises about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 15% w/w, or about 20% w/w of a betaine, including all values and ranges within these ranges. In some embodiments, the betaine is amidopropyl betaine, e.g., cocamidopropyl betaine.

Suitably, the shaving composition comprises about 0.01% w/w to about 20% w/w, preferably about 0.05% w/w to about 15% w/w, or more preferably about 0.1% w/w to about 10% w/w of a sarcosinate. In some embodiments, the shaving composition comprises about 0.01% w/w, about 0.03% w/w, about 0.05% w/w, about 0.10% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 15% w/w, or about 20% w/w of a betaine, including all values and ranges within these ranges. In some embodiments, the sarcosinate is lauroyl sarcosinate, e.g., sodium lauroyl sarcosinate.

In some embodiments, the amphoteric surfactant comprises an amphodiacetate, a betaine, and a sarcosinate. In some embodiments, the shaving composition comprises about 2% w/w to about 10% w/w of an amphodiacetate, about 0.5% w/w to about 10% w/w of a betaine, and about 0.1% to about 10% w/w of a sarcosinate. In further embodiments, the amphodiacetate is disodium cocoamphodiacetate. In yet further embodiments, the betaine is amidopropyl betaine. In still further embodiments, the sarcosinate is lauroyl sarcosinate, e.g., sodium lauroyl sarcosinate.

Suitably, the shaving composition described herein comprises an anionic surfactant. An “anionic surfactant” includes a negative charge (anionic group) on its hydrophilic head group. In some embodiments, the anionic group comprises a sulfonate, an ether sulfonate, a sulfate, an ether sulfate, a carboxylate, an ether carboxylate, a phosphate, an isethionate, a taurate, an amino acid surfactant, or a combination thereof.

In embodiments, the shaving composition comprises about 0.1% w/w to about 50% w/w, about 0.2% w/w to about 30% w/w, about 0.5% w/w to about 15% w/w, about 0.8% w/w to about 12% w/w, or about 1% w/w to about 10% w/w of the anionic surfactant. Suitably, the shaving composition comprises about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, about 30% w/w, about 35% w/w, about 40% w/w, about 45% w/w, or about 50% w/w of the anionic surfactant, including all values and ranges within these ranges.

Non-limiting examples of anionic surfactants include sulfonates, sulfates, carboxylates, isethionates, taurates, phosphates, citrates, galactouronates, sulfosuccinates, anionic alkylpolyglucoside derivatives, and amino acid surfactants such as alaninates, acylglutamates, aspartates, and glycinates. In some embodiments, the anionic surfactant of the shaving composition described herein comprises a sulfonate, a sulfate, an ether sulfate, an ether sulfonate, an ether carboxylate, an isethionate, a taurate, an amino acid surfactant, or a combination thereof.

Sulfonates may include alkylbenzene sulfonates (e.g., sodium dodecylbenzene sulfonate), alkyl sulfonates (e.g., alpha olefin sulfonates such as C₁₂₋₁₄ olefin sulfonate, C₁₄₋₁₆ olefin sulfonate, C₁₄₋₁₈ olefin sulfonate, C₁₆₋₁₈ olefin sulfonate), and ether sulfonates (e.g., alkyl ether sulfonate, alkyl polyoxyethylene ether sulfonate, fatty alcohol ether sulfonate). Sulfates may include alkyl sulfates (e.g., ammonium lauryl sulfate, sodium lauryl sulfate, sodium myreth sulfate) and ether sulfates (e.g., alkyl ether sulfates such as ammonium lauryl ether sulfate, sodium lauryl ether sulfate, sodium pareth sulfate, zinc coceth sulfate, C₆₋₂₄ alkyl ether sulfate, C₁₂₋₁₄ alkyl ether sulfate, C₁₂₋₁₅ alkyl ether sulfate).

Carboxylates may include amide ether carboxylates (e.g., sodium laurylamido ether carboxylate), polyoxyethylenated carboxylic acid salts (e.g., sodium lauryl ether carboxylate, polyoxyethylenated fatty acids of olive oil and of carboxymethyl, oxyethylenated sodium tridecyl ether carboxylate). Acetates may include sodium 2-(2-hydroxyalkyloxy)acetate. Phosphates may include monoalkyl phosphates and dialkyl phosphates (e.g., lauryl monophosphate, dodecylphosphate monoester, diester, or mixture thereof, octylphosphoric acid monoester, diester, or mixture thereof, ethoxylated 2-butyloctanol phosphoric monoester, diester, or mixture thereof).

Isethionates are esters of long-chain aliphatic carboxylic acids (e.g., C₈-C₁₈) with isethionic acid (2-hydroxyethanesulfonic acid). Exemplary isethionates include butyl isethionate, capryloyl isethionate, caproyl isethionate, lauroyl isethionate, and palmitoyl isethionate. Taurates, also termed taurides, comprise N-methyltaurine and a long-chain aliphatic carboxylic acid (e.g., C₈-C₁₈) linked via an amide bond. Exemplary taurates include methyl oleoyl taurate, methyl cocoyl taurate, and methyl stearyl taurate.

Amino acid surfactants refer to a class of surfactants obtained by condensation of natural amino acids with fatty acids or derivatives thereof, including alaninates, glutamates, acylglutamates, aspartates, and glycinates. Exemplary alaninates include sodium N-lauroyl-Nmethylamidopropionate and N-lauroyl N-methylalanine triethanolamine. Exemplary glutamates include cocoyl glutamate and lauroyl glutamate, Exemplary acylglutamates include triethanolamine monococoylglutamate and triethanolamine lauroylglutamate. Exemplary aspartates include triethanolamine N-lauroyl aspartate, triethanolamine N-myristoyl aspartate, and mixtures thereof. Exemplary glycinates include sodium N-cocoylglycinate.

Exemplary citrates include oxyethylenated (9 EO) citric monoester of coco alcohols. Exemplary galactouronates include sodium dodecyl-D-galactoside uronate. Exemplary anionic alkylpolyglucoside derivatives include glyceryl citrates, tartrates, sulfosuccinates, carbonates, and ethers obtained from alkyl polyglucosides, e.g., cocoylpolyglucoside (1,4) tartaric ester, cocoylpolyglucoside (1,4) sulfosuccinic ester, cocoylpolyglucoside (1,4) citric ester, and lauryl polyglucoside ether carboxylate.

Suitably, the shaving composition comprises about 0.1% w/w to about 30% w/w, preferably about 0.5% w/w to about 20% w/w, or more preferably about 1% w/w to about 15% w/w of an olein sulfonate. In some embodiments, the shaving composition comprises about 0.5% w/w, about 1% w/w, about 1.5% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 15% w/w, or about 20% w/w of an olefin sulfonate, including all values and ranges within these ranges. In some embodiments, the olefin sulfonate is C₁₂₋₁₄ olefin sulfonate, C₁₄₋₁₆ olefin sulfonate, C₁₄₋₁₈ olefin sulfonate, C₁₆₋₁₈ olefin sulfonate, or a combination thereof.

Suitably, the shaving composition described herein comprises a humectant. As used herein, “humectant” refers to a substance that attracts and/or retains moisture on a skin surface. Non-limiting examples of humectants include acetamide MEA, agarose, agave extract, algae extract, allantoin, aloe vera extract, ammonium lactate, arginine PCA, castor oil, collagen, copper PCA, corn glycerides, diglycereth-7 malate, diglycerin, dimethyl imidazolidinone, erythritol, gelatin, glucose, glucuronic acid, glucuronolactone, glutamic acid, glycereth-12, glycerin, glyceryl triacetate, goat milk, hexylene glycol, honey extract, hyaluronic acid, hydrolyzed wheat starch, hydroxyethyl sorbitol, lactamide, melibiose, panthenol, pantolactone, PCA, polyethylene glycol (PEG), polyglucuronic acid, polyglycerylmethacrylate, polypropylene glycol (PPG), saccharide hydrolysate, seaweed extract, sesame amino acids, sodium aspartate, sodium hexametaphosphate, sodium lactate, sodium malate, sodium PCA, sodium polyaspartate, TEA-lactate, triglycereth-7 citrate, urea, xylose, alpha hydroxy acids such as lactic acid, ethoxylated cocoates such as PEG-7 cocoate and PEG-15 cocoate, fatty acid glycerides such as cocoglyceride, glycols such as butylene glycol and propylene glycol, polymeric polyols such as polydextrose, proteins such as keratin, collagen, elastin, and silk protein, substituted diols such as 1,3-propanediol, and sugar alcohols such as glycerol, sorbitol, xylitol, and maltitol.

In embodiments, the shaving composition comprises about 0.05% w/w to about 30% w/w, preferably about 0.1% w/w to about 20% w/w, or more preferably about 0.1% w/w to about 15% w/w of the humectant. Suitably, the shaving composition comprises about 0.05% w/w, 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, about 10% w/w, about 15% w/w, about 20% w/w, about 25% w/w, or about 30% w/w of the humectant, including all values and ranges within these ranges.

Suitably, the shaving composition described herein comprises a moisturizing agent. As used herein, “moisturizing agent” or “moisturizer” refers to a substance that increases the water content of the skin surface, which can smooth and soften the skin, resulting in a more pliable and less flaky skin texture. Moisturizing agents can include occlusive agents, which slow the evaporation of water from the surface of the skin, and emollients, which remain in the outer surface of the skin to act as lubricants.

Non-limiting examples of occlusive agents include acetylated castor oil, acetylated lanolin alcohol, behenyl isostearate, beeswax, C₁₂₋₁₈ triglyceride, C₁₀₋₁₈ triglyceride, C₂₀₋₄₀ alcohol, C₁₆₋₃₆ alkyl stearate, C₁₈₋₇₀ isoparaffin, C₂₀₋₄₀ olefin, candelilla, canola oil, caprylic/capric triglycerides, carnauba, cetyl ricinoleate, cholesteryl oleate, decyl myristate, distearyl ether, glycol dioleate, hexyldecyl isosterate, hydrogenated castor oil, hydrogenated lanolin, isocetyl myristate, lanolin linoleate, lauryl cocoate, lecithin, myristyl myristate, neatsfool oil, octyldodecyl stearate, oleyl linoleate, palm kernel wax, paraffin, pentaerythrityl tetracocoate, petrolatum, propylene glycol dioleate, shark liver oil, soybean lipid, stearyl stearate, squalene, tall oil, tocopherol, trihexyldecyl citrate, and triisostearin.

Non-limiting examples of emollients include acetylated lanolin, acetyl trihexyl citrate, aloe vera and extracts thereof, argan oil, avocado sterols, butyl myristate, C₁₄₋₁₅ alcohols, C₁₂₋₁₃ alkyl ethylhexanoate, caprylyl glycol, castor oil, cetyl acetate, cetyl oleate, C₁₄₋₁₆ glycol palmitate, C₁₂₋₂₀ isoparaffin, cocoa butter, decyl oleate, diethylhexyl adipate, diethylhexyl malate, diisodecyl adipate, diisopropyl dilinoleate, dipropyl adipate, ethylhexyl palmitate, ethyl linoleate, glyceryl dioleate, glycyeryl linoleate, glyceryl ricinoleate, glyceryl stearate, glycol palmitate, glycol stearate, grape seed oil, hexyl laurate, isocetyl alcohol, isodecyl stearate, isohexyl palmitate, isopropyl isostearate, isopropyl myristate, isopropyl palmitate, isostearyl alcohol, jojoba oil, lanolin, methyl palmitate, myristyl propionate, natural fruits extracts such as cucumber extract, octyl octanoate, octyl stearate, PEG-4 lanolate, PEG-5 tristearyl citrate, PEG-7 glyceryl cocoate, polyglyceryl-6 oleate, polyglycerol-2 triisotearate, PPG-10 cetyl ether, PPG-11 stearyl ether, PPG-20 cetyl ether, PPG-4 laureth-2, propylene glycol linoleate, shea butter, squalene, sucrose oleate, sunflower seed oil glycerides, tall oil glycerides, tridecyl stearate, wheat germ glycerides, diglycerides and triglycerides such as lecithin, capric/caprylic triglycerides, PEG-10 soy sterol, and plant oils.

In embodiments, the shaving composition comprises about 0.01% w/w to about 25% w/w, preferably about 0.01% w/w to about 15% w/w, or more preferably about 0.01% w/w to about 5% w/w of the moisturizing agent. Suitably, the shaving composition comprises about 0.01% w/w, about 0.05% w/w, 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, about 10% w/w, about 15% w/w, about 20% w/w, or about 25% w/w of the moisturizing agent, including all values and ranges within these ranges. In certain embodiments, the shaving composition comprises 0% of the moisturizing agent, i.e., the composition does not comprise a moisturizing agent.

Suitably, the shaving composition described herein comprises one or more of a chelating agent, a preservative, a natural extract, an opacifier, a fluorinated foaming agent, and a fragrance. In some embodiments, each of the chelating agent, preservative, natural extract, opacifier, fluorinated foaming agent, and/or fragrance is present in an amount of about 0.001% w/w to about 20% w/w, or about 0.005% w/w to about 18% w/w, or about 0.01% w/w to about 15% w/w, or about 0.05% w/w to about 10% w/w, or about 1% w/w to about 5% w/w, or about 0.001% w/w to about 1% w/w, or about 0.01% w/w to about 1% w/w, or about 0.05% to about 5% w/w of the shaving composition. In some embodiments, each of the chelating agent, preservative, natural extract, opacifier, fluorinated foaming agents and/or fragrance is present in an amount of about 0.001% w/w, about 0.005% w/w, about 0.01% w/w, about 0.05% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 15% w/w, or about 20% w/w of the shaving composition, including all values and ranges within these ranges.

In some embodiments, the shaving composition described herein further comprises a chelating agent. Chelating agents bind to metal ions or metallic compounds and can prevent microbial contamination. Non-limiting examples of chelating agents suitable for the compositions described herein include EDTA, phytates and phytic acids such as sodium phytate/phytic acid, citrates and citric acids such as sodium citrate/citric acid, gluconates such as sodium gluconate, nitrilotriacetic acid (NTA) and salts thereof, ethylenediamine derivatives including trisodium ethylenediamine disuccinate (EDDS) and tetrahydroxypropyl ethylenediamine, succinic acid and salts thereof, cyclodextrin, activated carbon, and naturally-derived chelating agents such as DERMOFEEL® PA, DERMOFEEL® PA-3, and DERMOFEEL® PA-12.

In further embodiments, the shaving composition described herein further comprises a preservative. Preservatives prevent degradation of and/or prevent microbial growth in the composition. Non-limiting examples of preservatives suitable for the compositions described herein include benzoates and benzoic acids such as sodium benzoate, benzoate derivatives such as methyl benzoate, ethyl benzoate, butyl benzoate, and the like, phenoxyethanol, propylene glycol, methylchloroisothiazolinone, methylisothiazolinone, DMDM hydantoin, iodopropionylbutyl carbamate, salicylic acid, antioxidants such as vitamin E, rosemary oil extract, grapefruit seed extract, willow bark extract, and preservative formulations marketed under the trade names GERMABEN® II, LIQUAPAR™ oil, BENZOSTAT™, EUXYL® K 903, EUXYL® PE 9010, and GEOGARD®.

In still further embodiments, the shaving composition described herein further comprises a natural extract. The natural extract may be included to enhance the scent and/or skin feel properties of the composition. Exemplary natural extracts suitable for the compositions described herein include, but are not limited to, aloe extract, althea root extract, bergamot extract, calendula extract (marigold), chamomile extract, comfrey extract, cucumber extract, elderberry extract, fenugreek extract, flax seed extract, geranium extract, ginseng root extract, lavender extract, melon extract, milk thistle extract, mint extract (spearmint), oakmoss extract, oatmeal extract, rosemary extract, sage extract, sandalwood extract, slippery elm bark extract, St. John's wort extract, watercress extract, witch hazel extract, yarrow extract, and yucca extract.

In still further embodiments, the shaving composition described herein further comprises an opacifier. Opacifiers can be added to a transparent or translucent composition to render the composition more impervious to visible light. Non-limiting examples of opacifiers suitable for the compositions described herein include glycol stearate, glycol distearate, cetyl alcohol, cetostearyl alcohol, stearyl alcohol, distearyl ether, PEG distearates such as PEG-150 stearate, and metal oxides such as titanium dioxide.

In still further embodiments, the shaving composition further comprises a fluorinated foaming agent. The fluorinated foaming agent can be a low vapor pressure compound, which aids in the foaming of the shaving composition and/or improves the foaming characteristics of the shaving composition. For example, the fluorinated foaming agent can be polytetrafluoroethylene (PTFE) or a fluorosurfactant containing a perfluoroalkyl group, such as NOVEC™ FC-4430, FC-4432, and FC-4434 from 3M™ and ZONYL FSN-100 from DUPONT™.

In still further embodiments, the shaving composition described herein further comprises a fragrance. The fragrance may be a synthetic fragrance, a natural fragrance (e.g., a natural aromatic extract), an essential water, or a combination thereof.

In certain embodiments, the shaving composition further comprises a cationic component. The cationic component may improve the lubricity and feel of the skin. In some embodiments, the cationic component comprises a pH-dependent primary, secondary, or tertiary amine, such as octenidine dihydrochloride; or a quaternary ammonium salt, such as cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB). In additional embodiments, the cationic component comprises a cationic polymer, e.g., Polyquaternium-5, Polyquaternium-6, Polyquaternium-7, Polyquaternium-10, Polyquaternium-11, Polyquaternium-15, Polyquaternium-16, Polyquaternium-22, Polyquaternium-24, Polyquaternium-28, Polyquaternium-29, Polyquaternium-39, Polyquaternium-44, Polyquaternium-46, or Polyquaternium-47; or a cationic guar.

Suitably, the shaving composition comprises about 0.05% w/w to about 5% w/w, preferably about 0.05% w/w to about 1% w/w, or more preferably about 0.1% w/w to about 1% w/w of the cationic component. In some embodiments, the shaving composition comprises about 0.01% w/w, about 0.05% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.5% w/w, about 3% w/w, about 3.5% w/w, about 4% w/w, about 4.5% w/w, or about 5% w/w of the cationic component, including all values and ranges within these ranges.

Further exemplary components that can be included in the shaving composition described herein include, but are not limited to, a refreshing agent, a calmative, a vitamin, a filler, a dye, an antioxidant, an antibacterial agent, an antifungal agent, a propellant, or a combination thereof.

Suitably, the remainder (i.e., q.s.) of the shaving composition described herein is purified, sterilized, and/or deionized water. It is routine for one of ordinary skill in the art to determine the required weight percent of water necessary to make up the remainder of the composition so as to yield 100% by weight, taking into account the amounts of the other components, e.g., as described herein.

In some embodiments, the shaving composition described herein has a pH of about 3.5 to about 8, or preferably about 4.5 to about 7.5, or more preferably about 5.5 to about 7. Suitably, the pH of the shaving composition can be about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, or about 8. One of ordinary skill in the art will understand that a pH adjuster may be added to the composition to achieve the desired pH. The pH adjuster can be an acid, such acetic acid, citric acid, or lactic acid, or a base, such as sodium bicarbonate, sodium hydroxide, magnesium hydroxide, or arginine.

As described herein, the shaving composition are soap-free, e.g., comprising less than 1% w/w of a fatty acid, which provide substantially less dry skin as compared to soap-containing shaving products. Moreover, as described herein, the shaving composition lubricates the skin sufficiently during application and after shaving, such that additional lubricating agents are not required, and the shaving razors and devices do not require self-lubricating mechanisms during the shaving process. Thus, an additional lubricating agent can be included in the shaving compositions provided herein, but are not required. Such additional lubricating agents can be, for example:
cellulose or natural polymers, such as starch, xanthan or guar gum, carrageenan, alginates, polysaccharides, pectin, gelatin, and agar;
silicone oils, polymers, or copolymers thereof, such as dimethicone, cyclomethicone, polysilicone, and cyclopentasiloxane; and
mineral, vegetable, or sebaceous oils, such as coconut oil, olive oil, soybean oil, avocado oil, and palm oil.

In further embodiments, the shaving compositions herein do not comprise a gelling agent to modify the viscosity of the composition. Exemplary gelling agents that can be excluded are agents such as, but are not limited to, CARBOPOL® polyacrylic acid polymers, hydroxyalkylcellulose polymers such as hydroxyethylcellulose or hydroxypropylcellulose, carboxymethylcellulose and cellulose methyl ether, natural or synthetic gums such as xanthan gum, and starches.

Accordingly, in embodiments, the shaving composition described herein comprises less than 1% w/w of each of a cellulose or a natural polymer; a silicone oil, polymer, or copolymer thereof; a mineral, vegetable, or sebaceous oil; and a gelling agent. In a preferred embodiment, the shaving composition described herein comprises less than 0.5% w/w of each of a fatty acid; a cellulose or the natural polymer; a silicone oil, polymer, or copolymer thereof; a mineral, vegetable, or sebaceous oil; and a gelling agent. In a further preferred embodiment, the shaving composition described herein comprises less than 0.1% w/w of each of a fatty acid; a cellulose or natural polymer; a silicone oil, polymer, or copolymer thereof; a mineral, vegetable, or sebaceous oil; and a gelling agent. In further embodiments, the shaving composition does not comprise (i.e., is free of) a fatty acid; a cellulose or natural polymer; a silicone oil, polymer, or copolymer thereof; a mineral, vegetable, or sebaceous oil; or a gelling agent.

In further embodiments, the disclosure provides a shaving composition comprising:
a) about 1% w/w to about 15% w/w of an amphodiacetate;
b) about 0.1% w/w to about 15% w/w of an amidopropyl betaine;
c) about 0.05% w/w to about 15% w/w of lauroyl sarcosinate;
d) about 0.5% w/w to about 15% w/w of an olefin sulfonate;
e) about 0.05% w/w to about 20% w/w of a humectant;
f) 0% to about 5% w/w of a moisturizing agent; optionally one or more of a chelating agent; a preservative; a natural extract, an opacifier, a fluorinated foaming agent, and a fragrance; and
g) the remainder water,
wherein the composition has a pH of about 5.5 to about 7.5; and
wherein the composition comprises less than 1% w/w of each of: a fatty acid; a cellulose or a natural polymer; a silicone oil, polymer, or copolymer thereof, a mineral, vegetable, or sebaceous oil; and a gelling agent.

Suitably, the shaving composition comprises about 2% w/w to about 10% w/w of the amphodiacetate, e.g., about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, or about 10% w/w of the amphodiacetate. In embodiments, the amphodiacetate comprises disodium cocoamphodiacetate.

Suitably, the shaving composition comprises about 0.5% w/w to about 10% w/w of the amidopropyl betaine, e.g., about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, or about 10% w/w of the amidopropyl betaine. In embodiments, the amidopropyl betaine comprises cocamidopropyl betaine.

Suitably, the shaving composition comprises about 0.1% w/w to about 10% w/w of the lauroyl sarcosinate, e.g., about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, or about 10% w/w of the lauroyl sarcosinate. In embodiments, the lauroyl sarcosinate comprises sodium lauroyl sarcosinate.

Suitably, the shaving composition comprises about 1% w/w to about 10% w/w of the olefin sulfonate, e.g., about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, or about 10% w/w of the olefin sulfonate. In embodiments, the olefin sulfonate comprises a C₁₂₋₁₄ olefin sulfonate, a C₁₄₋₁₆ olefin sulfonate, a C₁₄₋₁₈ olefin sulfonate, a C₁₆₋₁₈ olefin sulfonate, or a combination thereof.

Suitably, the shaving composition comprises about 0.05% w/w to about 20% of the humectant, e.g., about 0.05% w/w, 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 1.5% w/w, about 2% w/w, about 2.5% w/w, about 3% w/w, about 4% w/w, about 5% w/w, about 6% w/w, about 7% w/w, about 8% w/w, about 9% w/w, about 10% w/w, about 15% w/w, or about 20% w/w of the humectant. Humectants are further described herein.

Suitably, each of the moisturizing agent, chelating agent, preservative, natural extract, opacifier, fluorinated foaming agent, and fragrance is about 0% to about 20% w/, about 0.01% w/w to about 20% w/w, or about 0.05% w/w to about 10% w/w, about 1% w/w to about 5% w/w, about 0.01% w/w to about 1% w/w, or about 0.05% to about 5% w/w of the shaving composition. Moisturizing agents, chelating agents, preservatives, natural extracts, opacifiers, fluorinated foaming agents, and fragrances are further described herein.

In further embodiments, the shaving composition comprises less than 0.5% w/w, or preferably less than 0.2% w/w, or more preferably less than 0.1% w/w of each of the fatty acid; the cellulose or the natural polymer; the silicone oil, polymer, or copolymer thereof, the mineral, vegetable, or sebaceous oil; and the gelling agent.

In some embodiments, the shaving composition does not comprise a fatty acid selected stearic acid, palmitic acid, and myristic acid. In further embodiments, the shaving composition does not comprise a cellulose or natural polymer selected from starch, xanthan or guar gum, carrageenan, alginates, polysaccharides, pectin, gelatin, and agar. In yet further embodiments, the shaving composition does not comprise a silicone oil, polymer, or copolymer thereof selected from dimethicone, cyclomethicone, polysilicone, and cyclopentasiloxane. In yet further embodiments, the shaving composition does not comprise a mineral, vegetable, or sebaceous oil selected from coconut oil, olive oil, soybean oil, avocado oil, and palm oil. In yet further embodiments, the shaving composition does not comprise a gelling agent xanthan gum, CARBOPOL® polyacrylic acid polymers, hydroxyalkylcellulose polymers such as hydroxyethylcellulose or hydroxypropylcellulose, carboxymethylcellulose and cellulose methyl ether, natural or synthetic gums, and starches.

Suitably, the pH of the shaving composition is about 5.5 to about 7.5, preferably about 5.5 to about 7. In some embodiments, the pH of the shaving composition is about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, or about 7.5. Suitably, the shaving composition further comprises a pH adjuster as described herein.

In further embodiments, the disclosure provides a shaving composition consisting of
a) about 1% w/w to about 50% w/w of an amphoteric surfactant;
b) about 0.1% w/w to about 20% w/w of an anionic surfactant;
c) about 0.1% w/w to about 20% w/w of a humectant;
d) one or more of a moisturizing agent, a chelating agent, a preservative, and a natural extract, an opacifier, a fluorinated foaming agent, and a fragrance; and
e) the remainder water,
wherein the composition has a pH of about 5.5 to about 7.5.

Suitably, the amphoteric surfactant is about 1% w/w to about 50% w/w, about 2% w/w to about 45% w/w, about 2.5% w/w to about 40% w/w, about 3% w/w to about 35% w/w, or about 5% w/w to about 30% w/w of the composition. Amphoteric surfactants are further described herein. Suitably, the amphoteric surfactant comprises an amphoacetate, an amphodiacetate, a betaine, a sarcosinate, a hydroxysultaine, or a combination thereof. In embodiments, the amphoteric surfactant comprises an amphodiacetate, e.g., disodium cocoamphodiacetate, a betaine, e.g., amidopropyl betaine, and a sarcosinate, e.g., lauroyl sarcosinate.

Suitably, the anionic surfactant is about 0.1% w/w to about 20% w/w, about 0.2% w/w to about 18% w/w, about 0.50% w/w to about 15% w/w, about 0.8% w/w to about 12% w/w, or about 1% w/w to about 10% w/w of the composition. Anionic surfactants are further described herein. Suitably, the anionic surfactant comprises a sulfonate, a sulfate, an ether sulfate, an ether sulfonate, an ether carboxylate, an isethionate, a taurate, an amino acid surfactant, or a combination thereof. In embodiments, the anionic surfactant comprises an olefin sulfonate, e.g., C₁₂₋₁₄ olefin sulfonate, C₁₄₋₁₆ olefin sulfonate, C₁₄₋₁₈ olefin sulfonate, or C₁₆₋₁₈ olefin sulfonate. In a preferred embodiment, the anionic surfactant comprises C₁₄₋₁₆ olefin sulfonate.

Suitably, the humectant is about 0.1% w/w to about 20% w/w, about 0.2% w/w to about 18% w/w, about 0.5% w/w to about 15% w/w, about 0.8% w/w to about 12% w/w, or about 1% w/w to about 10% w/w of the composition. Humectants are further described herein.

Suitably, each of the moisturizing agent, chelating agent, preservative, natural extract, opacifier, fluorinated foaming agent, and fragrance is about 0% to about 20% w/, about 0.01% w/w to about 20% w/w, or about 0.05% w/w to about 10% w/w, about 1% w/w to about 5% w/w, about 0.01% w/w to about 1% w/w, or about 0.05% to about 5% w/w of the shaving composition. Moisturizing agents, chelating agents, preservatives, natural extracts, opacifiers, fluorinated foaming agents, and fragrances are further described herein.

In some embodiments, the shaving composition described herein is a foam or a foamable composition. As used herein, the term “foam” or “foamable” as it relates to a composition means that the composition, upon actuation with an aerosol, produces a stabilized, homogenous, expandable composition that breaks easily upon shearing. The terms “foam” and “foamable composition” are used interchangeably throughout. Suitably, the foams and foamable compositions described herein are for application to a skin surface of a mammal, suitably a human.

In further embodiments, the disclosure provides a method of dispensing the shaving composition described herein, comprising:
a. providing the shaving composition in a gas-in-can dispenser, a finger-actuated dispenser, an aerosol dispenser, or a bag-on-valve dispenser; and
b. dispensing the shaving composition.

Suitably, the shaving composition is dispensed as a foam. In some embodiments, the dispenser is a monoblock aerosol device, i.e., containing only one compartment in which the composition is contained. In additional embodiments, different components of the composition are contained in separate compartments of the dispenser, and the different components are mixed together upon actuation of the dispenser.

In further embodiments, the disclosure provides a kit comprising: the shaving composition described herein, which can optionally be contained in a dispenser described herein; and a razor (e.g., a disposable razor) or shaving blade. In yet further embodiments, the kit further comprises a means for spreading the shaving composition, for example, a spatula. In yet further embodiments, the kit further comprises a lotion and/or an aftershave composition, e.g., to soothe razor burn.

All references cited herein, including patents, patent applications, papers, textbooks and the like, and the references cited therein, to the extent that they are not already, are hereby incorporated herein by reference in their entirety.

### Example

An exemplary shaving composition is produced according to Table 1:

| Component | Amount (w/w) |
|---|---|
| Amphodiacetate | 2.0-10.0% |
| Amidopropyl Betaine | 0.5-10.0% |
| Olefin Sulfonate | 1.0-10.0% |
| Lauroyl Sarcosinate | 0.1-10.0% |
| Humectant | 0.1-15.0% |
| Moisturizing Agent | 0.0-5.0% |
| Chelating Agent | Optional |
| Natural Extract | Optional |
| Preservative | q.s. |
| Water | q.s. |

The pH of the composition is adjusted to 3.5 to 7.5, or to 5.5 to 7.5.

The formulation is dispensed using a finger-actuated dispenser such as a foaming head pump; a gas-in-can, finger-actuated aerosol dispenser; or a bag-on-valve dispenser.

What is claimed is:

1. A shaving composition comprising:
a) about 1% w/w to about 50% w/w of an amphoteric surfactant;
b) about 0.1% w/w to about 20% w/w of an anionic surfactant;
c) about 0.1% w/w to about 20% w/w of a humectant;
d) 0% to about 15% w/w of a moisturizing agent;
e) optionally one or more of a chelating agent, a preservative, a natural extract, an opacifier, a fluorinated foaming agent, and a fragrance; and
f) the remainder water,
wherein the composition has a pH of about 3.5 to about 8, and
wherein the composition comprises less than 1% w/w of a fatty acid.

2. The shaving composition of claim 1, comprising about 2.5% w/w to about 40% w/w of the amphoteric surfactant.

3. The shaving composition of claim 1 or 2, wherein the amphoteric surfactant comprises an amphoacetate, an amphodiacetate, a betaine, a sarcosinate, a hydroxysultaine, or a combination thereof.

4. The shaving composition of any one of claims 1 to 3, comprising about 2% w/w to about 10% w/w of an amphodiacetate.

5. The shaving composition of any one of claims 1 to 4, comprising about 0.5% w/w to about 10% w/w of a betaine.

6. The shaving composition of any one of claims 1 to 5, comprising about 0.1% w/w to about 10% w/w of a sarcosinate.

7. The shaving composition of any one of claims 1 to 6, comprising about 2% w/w to about 10% w/w of an amphodiacetate, about 0.5% w/w to about 10% w/w of an amidopropyl betaine, and about 0.1% w/w to about 10% w/w of lauroyl sarcosinate.

8. The shaving composition of any one of claims 1 to 7, comprising about 0.5% w/w to about 15% w/w of the anionic surfactant.

9. The shaving composition of any one of claims 1 to 8, wherein the anionic surfactant comprises a sulfonate, a sulfate, an ether sulfate, an ether sulfonate, an ether carboxylate, an isethionate, a taurate, an amino acid surfactant, or a combination thereof.

10. The shaving composition of any one of claims 1 to 9, comprising about 1% w/w to about 10% of an olefin sulfonate.

11. The shaving composition of claim 10, wherein the olefin sulfonate is C₁₂₋₁₄ olefin sulfonate, C₁₄₋₁₆ olefin sulfonate, C₁₄₋₁₈ olefin sulfonate, C₁₆₋₁₈ olefin sulfonate, or a combination thereof.

12. The shaving composition of any one of claims 1 to 11, further comprising a cationic component.

13. The shaving composition of any one of claims 1 to 12, wherein the composition has a pH of about 5.5 to about 7.0.

14. The shaving composition of any one of claims 1 to 13, wherein the composition comprises less than about 1% w/w of each of a cellulose or a natural polymer; a silicone oil, polymer, or copolymer thereof, a mineral, vegetable, or sebaceous oil; and a gelling agent.

15. The shaving composition of claim 14, wherein the composition comprises less than 0.1% w/w of each of the fatty acid; the cellulose or the natural polymer; the silicone oil, polymer, or copolymer thereof; the mineral, vegetable, or sebaceous oil; and the gelling agent.

16. A shaving composition comprising:
a) about 1% w/w to about 15% w/w of an amphodiacetate;
b) about 0.1% w/w to about 15% w/w of an amidopropyl betaine;
c) about 0.05% w/w to about 15% w/w of lauroyl sarcosinate;
d) about 0.5% w/w to about 15% w/w of an olefin sulfonate;
e) about 0.05% w/w to about 20% w/w of a humectant;
f) 0% to about 5% w/w of a moisturizing agent;
g) optionally one or more of a chelating agent; a preservative; a natural extract, an opacifier, a fluorinated foaming agent, and a fragrance; and
h) the remainder water,
wherein the composition has a pH of about 5.5 to about 7.5; and
wherein the composition comprises less than 1% w/w of each of: a fatty acid; a cellulose or a natural polymer; a silicone oil, polymer, or copolymer thereof; a mineral, vegetable, or sebaceous oil; and a gelling agent.

17. The shaving composition of claim 13, comprising about 2% w/w to about 10% w/w of the amphodiacetate.

18. The shaving composition of claim 13 or 14, comprising about 0.5% w/w to about 10% w/w of the amidopropyl betaine.

19. The shaving composition of any one of claims 13 to 15, comprising about 0.1% w/w to about 10% w/w of lauroyl sarcosinate.

20. The shaving composition of any one of claims 13 to 16, comprising about 1% w/w to about 10% w/w of the olefin sulfonate.

21. The shaving composition of any one of claims 16 to 20, wherein the composition comprises less than about 0.1% of each of the fatty acid; the cellulose or the natural polymer; the silicone oil, polymer, or copolymer thereof; the mineral, vegetable, or sebaceous oil; and the gelling agent.

22. A shaving composition consisting of:
a) about 1% w/w to about 50% w/w of an amphoteric surfactant;
b) about 0.1% w/w to about 20% w/w of an anionic surfactant;
c) about 0.1% w/w to about 20% w/w of a humectant;
d) one or more of a moisturizing agent, a chelating agent, a preservative, and a natural extract, an opacifier, a fluorinated foaming agent, and a fragrance; and
e) the remainder water,
wherein the composition has a pH of about 5.5 to about 7.5.

23. The shaving composition of claim 22, wherein the amphoteric surfactant is about 2.5% w/w to about 40% w/w of the composition.

24. The shaving composition of claim 22 or 23, wherein the anionic surfactant is about 1% w/w to about 10% w/w of the composition.

25. The shaving composition of any one of claims 22 to 24, wherein the humectant is about 0.1% to about 15% w/w of the composition.

26. A method of dispensing the shaving composition of any of claims 1 to 25, comprising:
a. providing the shaving composition in a gas-in-can dispenser, a finger-actuated dispenser, an aerosol dispenser, or a bag-on-valve dispenser; and
b. dispensing the shaving composition.
